(19) United States
(12) Patent Application Publication
(10) Pub. No.: US 2002/0168335 A1
(43) Pub. Date: Nov. 14, 2002

(54) Cosmetic composition comprising a wax and a polymer

(76) Inventor: Nathalie Collin, Sceuax (FR)

Correspondence Address: Thomas L. Irving, FINNEGAN, HENDERSON, FARABOW,, GARRETT & DUNNER, L.L.P., 1300 I Street, N.W., Washington, DC, 20005-3315, US

(21) Appl. No.: 10/012,052

(22) Filed: Dec. 11, 2001

Related U.S. Application Data
(60) Provisional application No. 60/280,848, filed on Apr. 3, 2001.

(30) Foreign Application Priority Data
Dec. 12, 2000 (FR) 00 16163

(51) Int. Cl. A61K 31/785

(52) U.S. Cl. 424/78.35

(57) ABSTRACT

A subject matter of the invention is a composition comprising, in a physiologically acceptable aqueous medium, a wax-in-water emulsion and at least one first polymer with a weight-average molecular mass of less than 100 000 comprising a) a polymer backbone having hydrocarbonaceous repeat units which are provided with at least one amide unit and at least one pendent fatty chain and/or at least one end fatty chain, which fatty chains are optionally functionalized, have from 6 to 120 carbon atoms and are bonded to these amide units.

The composition makes it possible to rapidly and thickly make up keratinous substances.

Application to making up and caring for keratinous substances, in particular in mascara.

The present invention relates to a composition comprising, in a physiologically acceptable medium, a wax and a polymer comprising a specific amide unit intended in particular for the cosmetics field. The invention also relates to a process for cosmetically making up or cosmetically caring for keratinous substances. The makeup or care composition and process according to the invention are intended more particularly for human keratinous substances, such as the skin (including the scalp), the nails or keratinous fibers, in particular substantially longitudinal fibers, such as the eyelashes, eyebrows and hair. The invention relates more especially to a mascara.

The composition according to the invention can be provided in the form of a composition for coating the eyelashes (in particular a mascara), an eyeliner, a product for the eyebrows, a product for the lips, a face powder or eyeshadow, a foundation, a product for making up the body, a concealer, a nail varnish, a product for caring for the skin, including the scalp, or a product for the hair (hair mascara, spray).

The makeup composition can also be applied to makeup accessories (support), such as false eyelashes, toupees, wigs or false nails, or to disks or patches which adhere to the skin or lips (of the beauty spot type).

Compositions for coating the eyelashes, known as mascaras, generally comprise, in a known way, at least one wax, in the form of a wax-in-water emulsion, and at least one film-forming polymer for depositing a makeup film on the eyelashes and sheathing the latter, as is disclosed, for example, in the document WO-A-95/15741. Users expect good cosmetic properties from these products, such as adhesion to the eyelashes, a lengthening or a curling of the eyelashes, or good hold of the mascara over time, in particular good resistance to rubbing, for example by the fingers or fabrics (handkerchiefs, serviettes).

However, with these compositions, the makeup properties, such as the sheathing, the elongation or the curling of the eyelashes are obtained when a significant amount of product is deposited on the eyelashes using an applicator, such as a mascara brush. When the composition does not adhere well to the eyelashes, the user has to apply the brush, impregnated with product, several times to the eyelashes, which requires the devotion of a degree of time in order to apply the makeup and to obtain the desired makeup results. In point of fact, this time may be perceived as far too long by users in a hurry. A need thus exists to have available mascaras which make it possible to rapidly and easily obtain the expected makeup.

The aim of the present invention is to have available a composition for making up keratinous substances, in particular keratinous fibers, such as the eyelashes, which is easily applied to keratinous substances and which rapidly results in a makeup exhibiting good cosmetic properties.

The inventors have found, surprisingly, that the use of a polymer comprising a specific amide unit in a composition comprising a wax-in-water emulsion makes it possible to improve the properties of adhesion of the composition to keratinous substances, in particular to keratinous fibers, such as the eyelashes. The composition is easily applied to keratinous substances and makes it possible to quickly deposit the composition in an amount sufficient to produce a makeup exhibiting the expected cosmetic properties. In particular, a thick layer of the makeup on keratinous substances is rapidly obtained, which saves the users the trouble of applying the composition to keratinous substances for an excessive length of time. Thus, for a mascara, a makeup is obtained which rapidly thickens keratinous fibers, in particular the eyelashes; instantaneous volumizing of the eyelashes is thus observed. Furthermore, the mascara confers good lengthening on the made-up eyelashes.

More specifically, a subject matter of the invention is a composition comprising, in a physiologically acceptable aqueous medium, a wax-in-water emulsion and at least one first polymer with a weight-average molecular mass of less than 100 000 comprising a polymer backbone having nonpendent amide units and at least one pendent fatty chain and/or at least one end fatty chain, which fatty chains are optionally functionalized, have from 6 to 120 carbon atoms and are bonded to these amide units.

Another subject matter of the invention is a cosmetic process for making up or caring for human keratinous substances, comprising the application, to the keratinous substances, of a composition as defined above. The process preferably applies to substantially longitudinal keratinous fibers, such as the eyelashes, the hair or eyebrows), and more especially to the eyelashes.

A further subject matter of the invention is the use of a composition as defined above for producing an adherent layer and/or fast making up on keratinous substances.

Another subject matter of the invention is the use of a mascara comprising a composition as defined above for rapidly thickening and/or for lengthening the eyelashes.

Another subject matter of the invention is the use of the combination of at least one first polymer with a weight-average molecular mass of less than 100 000 and better still of less than 50 000, comprising a) a polymer backbone having hydrocarbonaceous repeat units which are provided with at least one nonpendent amide unit and at least one pendent fatty chain and/or at least one end fatty chain, which fatty chains are optionally functionalized, have from 6 to 120 carbon atoms and are bonded to these amide units, and of at least one wax, in a physiologically acceptable composition in the form of a wax-in-water emulsion, for producing an adherent layer on keratinous substances and/or fast making up of keratinous substances and/or for rapidly thickening and/or for lengthening the eyelashes.

The term “physiologically acceptable medium” is understood to mean a nontoxic medium capable of being applied to the skin, superficial body growths or lips of human beings, such as a cosmetic medium.

The term “functionalized chains”, within the meaning of the invention, is understood to mean an alkyl chain comprising one or more functional or reactive groups chosen in particular from amide, hydroxyl, ether, oxyalkylene or polyoxyalkylene, halogen, ester, siloxane or polysiloxane groups, the halogen groups including fluorinated or perfluorinated groups. In addition, the hydrogen atoms of one or more fatty chains can be at least partially substituted by fluorine atoms.

According to the invention, these chains can be bonded directly to the polymer backbone or via an ester functional group or a perfluorinated group.

The term “polymer” is understood to mean, within the meaning of the invention, a compound having at least 2 repeat units and preferably at least 3 repeat units.

The term “hydrocarbonaceous repeat unit” is understood to mean, within the meaning of the invention, a unit which comprises from 2 to 80 carbon atoms and preferably from 2 to 60 carbon atoms, which carries hydrogen atoms and optionally oxygen atoms, and which can be linear, branched or cyclic and saturated or unsaturated. In addition, these units each comprise from one to several nonpendent amide units which are found in the polymer backbone.

The pendent chains are advantageously bonded directly to at least one of the amide units of the polymer backbone.

The first polymer can comprise, between the hydrocarbonaceous units, silicone units or oxyalkylenated units.

In addition, the first polymer of the composition of the invention advantageously comprises from 40 to 98% of fatty chains with respect to the total number of the amide units and of the fatty chains and better still from 50 to 95%. The proportion of the amide units depends on the nature of the fatty phase and is in particular similar to the polar nature of the fatty phase. Thus, the higher the proportion of amide units in the first polymer, which corresponds to the presence of several amide units, the greater affinity of the first polymer for polar oils.

Advantageously, the first polymer of the composition according to the invention exhibits a weight-average molecular mass of less than 100 000 (in particular ranging from 1 000 to 100 000), in particular of less than 50 000 (in particular ranging from 1 000 to 50 000), and more particularly ranging from 1 000 to 30 000, preferably from 2 000 to 20 000, and better still from 2 000 to 10 000.

The first polymer, and in particular the polyamide, is insoluble in water, in particular at 25° C. In particular, it does not comprise an ionic group.

Mention may be made, as preferred first polymers which can be used in the invention, of polyamides branched by pendent fatty chains and/or end fatty chains having from 6 to 120 carbon atoms and better still from 8 to 120 and in particular from 12 to 68 carbon atoms, each end fatty chain being bonded to the polyamide backbone via at least one bonding group, in particular an ester group. These polymers preferably comprise a fatty chain at each end of the polyamide backbone. Mention may be made, as other bonding group, of ether, amine, urea, urethane, thioester, thiurea or thiourethane groups.

These first polymers are preferably polymers resulting from a polycondensation between a dicarboxylic acid having at least 32 carbon atoms (in particular having from 32 to 44 carbon atoms) with an amine chosen from diamines having at least 2 carbon atoms (in particular from 2 to 36 carbon atoms) and triamines having at least 2 carbon atoms (in particular from 2 to 36 carbon atoms. The diacid is preferably a dimer resulting from a fatty acid comprising ethylenic unsaturation having at least 16 carbon atoms, preferably from 16 to 24 carbon atoms, such as oleic, linoleic or linolenic acid. The diamine is preferably ethylenediamine, hexylenediamine or hexamethylene-diamine. The triamine is, for example, ethylene-triamine. For polymers comprising one or 2 end carboxylic acid groups, it is advantageous to esterify them with a monoalcohol having at least 4 carbon atoms, preferably from 10 to 36 carbon atoms and better still from 12 to 24 and even better still from 16 to 24, for example 18 carbon atoms.

These polymers are more especially those disclosed in the document U.S. Pat. No. 5,783,657 of Union Camp. Each of these polymers satisfies in particular the following formula (I):

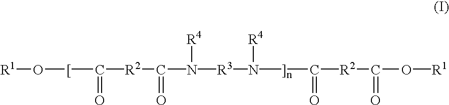

in which n denotes a whole number of amide units such that the number of ester groups represents from 10% to 50% of the total number of the ester and amide groups; R¹ is, in each case, independently an alkyl or alkenyl group having at least 4 carbon atoms and in particular from 4 to 24 carbon atoms; R² independently represents, in each case, a C₄ to C₄₂ hydrocarbonaceous group, provided that 50% of the R² groups represent a C₃₀ to C₄₂ hydrocarbonaceous group; R³ independently represents, in each case, an organic group provided with at least 2 carbon atoms, with hydrogen atoms and optionally with one or more oxygen or nitrogen atoms; and R⁴ independently represents, in each case, a hydrogen atom, a C₁ to C₁₀ alkyl group or a direct bond to R³ or to another R⁴, so that the nitrogen atom to which both R³ and R⁴ are bonded forms part of a heterocyclic structure defined by R⁴—N—R³, with at least 50% of the R⁴ groups representing a hydrogen atom.

In the specific case of the formula (I), the optionally functionalized end fatty chains within the meaning of the invention are end chains bonded to the final nitrogen of the polyamide backbone.

In particular, the ester groups of the formula (I), which form part of the end and/or pendent fatty chains within the meaning of the invention, represent from 15 to 40% of the total number of the ester and amide groups and better still from 20 to 35%. Furthermore, n advantageously represents a whole number ranging from 1 to 5 and better still of greater than 2. Preferably, R¹ is a C₁₂ to C₂₂ alkyl group and preferably a C₁₆ to C₂₂ alkyl group. Advantageously, R² can be a C₁₀ to C₄₂ hydrocarbonaceous (alkylene) group. Preferably, at least 50% and better still at least 75% of the R² groups are groups having from 30 to 42 carbon atoms. The other R² groups are C₄ to C₁₉ and even C₄ to C₁₂ hydrogenated groups. Preferably, R³ represents a C₂ to C₃₆ hydrocarbonaceous group or a polyoxyalkylenated group and R⁴ represents a hydrogen atom. Preferably, R³ represents a C₂ to C₁₂ hydrocarbonaceous group.

The hydrocarbonaceous groups can be linear, cyclic or branched and saturated or unsaturated groups. Furthermore, the alkyl and alkylene groups can be linear or branched and saturated or unsaturated groups.

In general, the polymers of formula (I) are provided in the form of blends of polymers, it being possible for these blends additionally to comprise a synthetic product corresponding to a compound of formula (I) where n has the value 0, that is to say a diester.

Mention may be made, as examples of first polymers according to the invention, of the commercial products sold by Arizona Chemical under the names Uniclear 80 and Uniclear 100. They are sold respectively in the form of an 80% (as active material) gel in a mineral oil and of a 100% (as active material) gel. They have a softening point of 88 to 94° C. These commercial products are a blend of copolymers of a C₃₆ diacid condensed with ethylenediamine, with a weight-average molecular mass of approximately 6 000. The end ester groups result from the esterification of the remaining acid endings with cetyl alcohol, stearyl alcohol or their mixtures (also known as cetearyl alcohol).

Mention may also be made, as first polymer which can be used in the invention, of polyamide resins resulting from the condensation of an aliphatic dicarboxylic acid and of a diamine (including compounds having more than 2 carbonyl groups and 2 amine groups), the carbonyl and amine groups of adjacent individual units being condensed via an amide bond. These polyamide resins are in particular those sold under the Versamid® trademark by General Mills Inc. and Henkel Corp. (Versamid 930, 744 or 1655) or by Olin Mathieson Chemical Corp. under the Onamid® trademark, in particular Onamid S or C. These resins have a weight-average molecular mass ranging from 6 000 to 9 000. For further information on these polyamides, reference may be made to documents U.S. Pat. No. 3,645,705 and U.S. Pat. No. 3,148,125. More especially, Versamid® 930 or 744 is used.

It is also possible to use the polyamides sold by Arizona Chemical under the Uni-Rez references (2658, 2931, 2970, 2621, 2613, 2624, 2665, 1554, 2623, 2662) and the product sold under the reference Macromelt 612 by Henkel. For further information on these polyamides, reference may be made to the document U.S. Pat. No. 5,500,209.

It is also possible to use polyamide resins resulting from vegetables, such as those disclosed in the patents U.S. Pat. No. 5,783,657 and U.S. Pat. No. 5,998,570.

The first polymer present in the composition according to the invention advantageously has a softening temperature of greater than 65° C. and which can range up to 190° C. It preferably exhibits a softening temperature ranging from 70 to 130° C. and better still from 80 to 105° C. The first polymer is in particular a nonwaxy polymer.

Preferably, the first polymer according to the invention corresponds to the abovementioned formula (I). This first polymer exhibit, because of its/their fatty chain(s), good solubility in oils and thus result in macroscopically homogeneous compositions, even with a high level (at least 25%) of polymer, in contrast to polymers without a fatty chain.

The first polymer can be present in the composition according to the invention in a content ranging from 0.01% to 10% by weight with respect to the total weight of the composition, preferably ranging from 0.05% to 5% by weight and better still ranging from 0.1% to 3% by weight.

The fatty phase of the composition according to the invention can comprise a wax. The term “wax” is understood to mean, within the meaning of the present invention, a lipophilic fatty compound which is solid at ambient temperature (25° C.) and atmospheric pressure (760 mm of Hg, i.e. 10⁵ Pa), with a reversible solid/liquid state change, having a melting temperature of greater than 30° C. and better still of greater than 55° C. which can range up to 200° C., in particular up to 120° C. By bringing the wax to its melting temperature, it is possible to render it miscible with the oils and to form a microscopically homogeneous mixture but, by bringing the temperature of the mixture back to ambient temperature, recrystallization of the wax in the oils of the mixture is obtained.

The melting point values correspond, according to the invention, to the melting peak measured using a differential scanning calorimeter (D.S.C.), for example the calorimeter sold under the name DSC 30 by Metler, with a temperature rise of 5 or 10° C. per minute.

The waxes within the meaning of the invention are those generally used in the cosmetics and dermatological fields. Mention may in particular be made of beeswax, lanolin wax and Chinese insect waxes; rice wax, carnauba wax, candelilla wax, ouricury wax, cork fiber wax, sugar cane wax, Japan wax and sumac wax; montan wax, microcrystalline waxes, paraffin waxes, ozokerites, ceresin wax, lignite wax, polyethylene waxes, waxes obtained by the Fischer-Tropsch synthesis, or fatty acid esters and glycerides which are solid at 40° C. and better still at more than 55° C.

Mention may also be made of the waxes obtained by catalytic hydrogenation of animal or vegetable oils having linear or branched C₈-C₃₂ fatty chains. Mention may in particular be made, among these, of hydrogenated jojoba oil, hydrogenated sunflower oil, hydrogenated castor oil, hydrogenated coconut oil and hydrogenated lanolin oil.

Mention may further be made of silicone waxes or fluorinated waxes.

The waxes present in the composition can be dispersed in the form of particles in an aqueous medium. These particles can have a mean size ranging from 50 nm to 10 μm and preferably from 50 nm to 3.5 μm.

The wax can in particular be present in the form of a waxes-in-water emulsion, it being possible for the waxes to be in the form of particles with a mean size ranging from 1 μm to 10 μm and preferably from 1 μm to 3.5 μm.

In another embodiment of the composition according to the invention, the wax can be present in the form of a wax microdispersion, the wax being in the form of particles with a mean size of less than 1 μm and ranging in particular from 50 nm to 500 nm. Wax micro-dispersions are disclosed in the documents EP-A-5 571 196 and EP-A-1 048 282.

The wax can also exhibit a hardness ranging from 0.05 MPa to 15 MPa and preferably ranging from 6 MPa to 15 MPa. The hardness is determined by the measurement of the compressive force, measured at 20° C. using the texture analyzer sold under the name TA-XT2i by Rhéo, equipped with a stainless steel cylinder with a diameter of 2 mm moving at the measurement speed of 0.1 mm/s and penetrating into the wax to a depth of penetration of 0.3 mm. To carry out the hardness measurement, the wax is melted at a temperature equal to the melting point of the wax +20° C. The molten wax is poured into a receptacle with a diameter of 30 mm and a depth of 20 mm. The wax is recrystallized at ambient temperature (25° C.) for 24 hours and then the wax is stored for at least 1 hour at 20° C. before carrying out the hardness measurement. The value of the hardness is the compressive force measured divided by the surface area of the cylinder of the texture analyzer in contact with the wax.

The wax can be present in the composition according to the invention in a content ranging from 0.1% to 50% by weight with respect to the total weight of the composition, preferably from 0.5% to 30% by weight and better still from 1% to 20% by weight.

Advantageously, the first polymer and the wax can be present in the composition according to the invention according to a wax/first polymer ratio by weight ranging from 5 to 60, preferably ranging from 7 to 50 and better still from 10 to 40.

The aqueous phase constitutes the continuous phase of the composition. The aqueous phase can be composed essentially of water; it can also comprise a mixture of water and of water-miscible solvent (miscibility in water of greater than 50% by weight at 25° C.), such as lower monoalcohols having from 1 to 5 carbon atoms, for example ethanol or isopropanol, glycols having from 2 to 8 carbon atoms, for example propylene glycol, ethylene glycol, 1,3-butylene glycol or dipropylene glycol, C₃-C₄ ketones or C₂-C₄ aldehydes. The water-miscible solvent can be present in a content ranging from 0.1% to 20% by weight with respect to the total weight of the composition and preferably ranging from 0.1% to 10% by weight. In particular, the content of water-miscible organic solvent represents from 0.1% to 30% of the weight of water present in the composition.

The aqueous phase (water and optionally the water-miscible organic solvent) can be present in a content ranging from 1% to 95% by weight with respect to the total weight of the composition, preferably from 5% to 80% by weight and better still from 10% to 60% by weight.

The composition according to the invention can comprise emulsifying surface-active agents present in particular in a proportion ranging from 2 to 30% by weight with respect to the total weight of the composition and better still from 5% to 15%. These surface-active agents can be chosen from anionic or nonionic surface-active agents. Reference may be made to the document “Encyclopedia of Chemical Technology, Kirk-Othmer”, volume 33, p. 333-432, 3rd edition, 1979, Wiley, for the definition of the properties and functions (emulsifying) of surfactants, in particular p. 347-377 of this reference for anionic and nonionic surfactants.

The surfactants preferably used in the composition according to the invention are chosen: —from nonionic surfactants: fatty acids, fatty alcohols, polyethoxylated or polyglycerolated fatty alcohols, such as polyethoxylated stearyl or cetearyl alcohols, esters of a fatty acid and of sucrose, alkyl glucose esters, in particular polyoxyethylenated fatty esters of C₁-C₆ alkyl glucose, and their mixtures; —from anionic surfactants: C₁₆-C₃₀ fatty acids neutralized with amines, aqueous ammonia or alkaline salts, and their mixtures.

Use is preferably made of surfactants which make it possible to obtain an oil-in-water or wax-in-water emulsion.

The composition according to the invention can comprise at least one second additional film-forming polymer other than the first polymer described above.

The second film-forming polymer can be present in the composition according to the invention in a content on a dry basis ranging from 0.1% to 60% by weight with respect to the total weight of the composition, preferably from 0.5% to 40% by weight and better still from 1% to 30% by weight.

In the present application, the term “film-forming polymer” is understood to mean a polymer capable of forming, by itself alone or in the presence of a subsidiary agent which is able to form a film, a continuous film which adheres to support, in particular to keratinous substances.

Use is preferably made of a film-forming polymer capable of forming a hydrophobic film, that is to say a polymer, the film of which has a solubility in water at 25° C. of less than 1% by weight.

Mention may be made, among film-forming polymers which can be used in the composition of the present invention, of synthetic polymers, of radical type or of polycondensate type, polymers of natural origin, and their mixtures.

The term “radical film-forming polymer” is understood to mean a polymer obtained by polymerization of monomers comprising unsaturation, in particular ethylenic unsaturation, each monomer being capable of homopolymerizing (unlike polycondensates). The film-forming polymers of radical type can in particular be vinyl polymers or copolymers, in particular acrylic polymers.

The vinyl film-forming polymers can result from polymerization of monomers comprising ethylenic unsaturation having at least one acid group and/or esters of these acid monomers and/or amides of these acid monomers.

Use may be made, as monomer carrying an acid group, of α,β-ethylenic unsaturated carboxylic acids, such as acrylic acid, methacrylic acid, crotonic acid, maleic acid or itaconic acid. Use is preferably made of (meth)acrylic acid and crotonic acid and more preferably of (meth)acrylic acid.

The esters of acid monomers are advantageously chosen from esters of (meth)acrylic acid (also known as (meth)acrylates), in particular alkyl (meth)acrylates, especially C₁-C₃₀ alkyl (meth)acrylates, preferably C₁-C₂₀ alkyl (meth)acrylates, aryl (meth)acrylates, in particular C₆-C₁₀ aryl (meth)acrylates, or hydroxyalkyl (meth)acrylates, in particular C₂-C₆ hydroxyalkyl (meth)acrylates. Mention may be made, among alkyl (meth)acrylates, of methyl methacrylate, ethyl methacrylate, butyl methacrylate, isobutyl methacrylate, 2-ethylhexyl methacrylate, lauryl methacrylate or cyclohexyl methacrylate.

Mention may be made, among hydroxyalkyl (meth)acrylates, of hydroxyethyl acrylate, 2-hydroxy-propyl acrylate, hydroxyethyl methacrylate or 2-hydroxypropyl methacrylate.

Mention may be made, among aryl (meth)acrylates, of benzyl acrylate and phenyl acrylate. The esters of (meth)acrylic acid which are particularly preferred are the alkyl (meth)acrylates.

According to the present invention, the alkyl group of the esters can be either fluorinated or perfluorinated, that is to say that a portion or all of the hydrogen atoms of the alkyl group are substituted by fluorine atoms.

Mention may for example be made, as amides of the acid monomers, of (meth)acrylamides and in particular N-alkyl(meth)acrylamides, especially with a C₂-C₁₂ alkyl. Mention may be made, among N-alkyl(meth)acrylamides, of N-ethylacrylamide, N-(t-butyl)acrylamide, N-(t-octyl)acrylamide and N-undecylacrylamide.

The vinyl film-forming polymers can also result from the homopolymerization or copolymerization of monomers chosen from vinyl esters and styrene monomers. In particular, these monomers can be polymerized with acid monomers and/or their esters and/or their amides, such as those mentioned above. Mention may be made, as examples of vinyl esters, of vinyl acetate, vinyl neodecanoate, vinyl pivalate, vinyl benzoate and vinyl t-butylbenzoate. Mention may be made, as styrene monomers, of styrene and α-methylstyrene.

It is possible to use any monomer known to a person skilled in the art coming within the categories of acrylic and vinyl monomers (including monomers modified by a silicone chain).

Mention may be made, among film-forming polycondensates, of polyurethanes, polyesters, polyesteramides, polyamides, and epoxyester resins, or polyureas.

The polyurethanes can be chosen from polyurethanes which are anionic, cationic, nonionic or amphoteric, polyurethane-acrylics, polyurethane-polyvinylpirrolidones, polyester-polyurethanes, polyether-polyurethanes, polyureas, polyurea-polyurethanes, and their mixtures.

The polyesters can be obtained, in a known way, by polycondensation of dicarboxylic acids with polyols, in particular diols.

The dicarboxylic acid can be aliphatic, alicyclic or aromatic. Mention may be made, as examples of such acids, of: oxalic acid, malonic acid, dimethylmalonic acid, succinic acid, glutaric acid, adipic acid, pimelic acid, 2,2-dimethylglutaric acid, azelaic acid, suberic acid, sebacic acid, fumaric acid, maleic acid, itaconic acid, phthalic acid, dodecanedioic acid, 1,3-cyclohexanedicarboxylic acid, 1,4-cyclohexane-dicarboxylic acid, isophthalic acid, terephthalic acid, 2,5-norboranedicarboxylic acid, diglycolic acid, thiodipropionic acid, 2,5-naphthalenedicarboxylic acid or 2,6-naphthalenedicarboxylic acid. These dicarboxylic acid monomers can be used alone or as a combination of at least two dicarboxylic acid monomers. Preferably, among these monomers, phthalic acid, isophthalic acid or terephthalic acid is chosen.

The diol can be chosen from aliphatic, alicyclic or aromatic diols. Use is preferably made of a diol chosen from: ethylene glycol, diethylene glycol, triethylene glycol, 1,3-propanediol, cyclohexane-dimethanol or 4-butanediol.

Use may be made, as other polyols, of glycerol, penta-erythritol, sorbitol or trimethylolpropane.

The polyesteramides can be obtained analogously to the polyesters by polycondensation of diacids with diamines or aminoalcohols. Use may be made, as diamine, of ethylenediamine, hexamethylene-diamine or meta- or para-phenylenediamine. Use may be made, as aminoalcohol, of monoethanolamine.

The polyester can additionally comprise at least one monomer carrying at least one —SO₃M group, with M representing a hydrogen atom, an ammonium ion NH₄⁺ or a metal ion, such as, for example, an Na⁺, Li⁺, K⁺, Mg²⁺, Ca²⁺, Cu²⁺, Fe²⁺ or Fe³⁺ ion. Use may in particular be made of a bifunctional aromatic monomer comprising such an —SO₃M group.

The aromatic nucleus of the bifunctional aromatic monomer additionally carrying an —SO₃M group as described above can be chosen, for example, from benzene, naphthalene, anthracene, diphenyl, oxydiphenyl, sulfonyldiphenyl or methylenediphenyl nuclei. Mention may be made, as examples of bifunctional aromatic monomer additionally carrying an —SO₃M group, of: sulfoisophthalic acid, sulfo-terephthalic acid, sulfophthalic acid or 4-sulfo-naphthalene-2,7-dicarboxylic acid.

It is preferable to use copolymers based on isophthalate/sulfoisophthalate and more particularly copolymers obtained by condensation of diethylene glycol, cyclohexanedimethanol, isophthalic acid and sulfoisophthalic acid. Such polymers are sold, for example, under the Eastman AQ® trademark by Eastman Chemical Products.

The optionally modified polymers of natural origin can be chosen from shellac resin, sanderac gum, dammars, elemis, copals, cellulose polymers, and their mixtures.

According to a first embodiment of the composition according to the invention, the second film-forming polymer can be present in the form of particles in aqueous dispersion, a form generally known under the name of latex or pseudolatex. The techniques for the preparation of these dispersions are well known to a person skilled in the art.

Use may be made, as aqueous dispersion of film-forming polymer, of the acrylic dispersions sold under the names Neocryl XK-90®, Neocryl A-1070®, Neocryl A-1090®, Neocryl BT-62®, Neocryl A-1079® or Neocryl A-523® by Avecia-Neoresins, Dow Latex 432® by Dow Chemical or Daitosol 5000 AD® by Daito Kasey Kogyo; or alternatively of the aqueous polyurethane dispersions sold under the names Neorez R-981® or Neorez R-974® by Avecia-Neoresins, Avalure UR-405®, Avalure UR-410®, Avalure UR-425®, Avalure UR-450®, Sancure 875®, Sancure 861®, Sancure 878® or Sancure 2060® by Goodrich, Impranil 85® by Bayer or Aquamere H-1511® by Hydromer.

Use may also be made, as aqueous dispersion of film-forming polymer, of the dispersions of polymers resulting from the radical polymerization of one or more radical monomers inside and/or partially at the surface of preexisting particles of at least one polymer chosen from the group consisting of polyurethanes, polyureas, polyesters, polyesteramides and/or alkyds. These polymers are generally known as hybrid polymers.

According to a second alternative embodiment of the composition according to the invention, the film-forming polymer can be a water-soluble polymer and is thus present in the aqueous phase of the composition in the dissolved form. Mention may be made, as examples of water-soluble film-forming polymers, of:

proteins, such as proteins of plant origin, such as wheat or soya proteins; or proteins of animal origin, such as keratins, for example keratin hydrolysates and sulfonic keratin;

anionic, cationic, amphoteric or nonionic chitosan or chitin polymers;

cellulose polymers, such as hydroxyethylcellulose, hydroxypropylcellulose, methylcellulose, ethylhydroxy-ethylcellulose or carboxymethylcellulose, and quaternized cellulose derivatives;

acrylic polymers or copolymers, such as polyacrylates or polymethacrylates;

vinyl polymers, such as polyvinylpyrrolidones, copolymers of methyl vinyl ether and of malic anhydride, the copolymer of vinyl acetate and of crotonic acid, copolymers of vinylpyrrolidone and of vinyl acetate; copolymers of vinylpyrrolidone and of caprolactam; or poly(vinyl alcohol);

optionally modified polymers of natural origin, such as:

gums arabic, guar gum, xanthan derivatives or karaya gum;

alginates and carrageenans;

glycoaminoglycans, hyaluronic acid and its derivatives;

shellac resin, sandarac gum, dammars, elemis or copals;

deoxyribonucleic acid;

mucopolysaccharides, such as hyaluronic acid, chondroitin sulfates, and their mixtures.

The size of the particles of polymers in dispersion in the aqueous phase can range from 5 nm to 600 nm and preferably from 20 nm to 300 nm.

The composition according to the invention can comprise a subsidiary agent which is able to form a film which promotes the formation of a film with the film-forming polymer. Such an agent which is able to form a film can be chosen from any compound known to a person skilled in the art as being capable of fulfilling the desired role and in particular can be chosen from plasticizing agents and coalescence agents.

The composition according to the invention can also comprise a coloring material, such as pulverulent coloring materials, fat-soluble dyes or water-soluble dyes. This coloring material can be present in a content ranging from 0.01% to 30% by weight with respect to the total weight of the composition.

The pulverulent coloring materials can be chosen from pigments and pearlescent agents.

The pigments can be white or colored, inorganic and/or organic and coated or uncoated. Mention may be made, among inorganic pigments, of titanium dioxide, which is optionally surface treated, zirconium, zinc or cerium oxides and iron or chromium oxides, manganese violet, ultramarine blue, chromium hydrate and ferric blue. Mention may be made, among organic pigments, of carbon black, pigments of D & C type and lakes based on cochineal carmine or on barium, strontium, calcium or aluminum.

The pearlescent agents can be chosen from white pearlescent pigments, such as mica covered with titanium oxide or with bismuth oxychloride, colored pearlescent pigments, such as titanium oxide-coated mica with iron oxides, titanium oxide-coated mica with in particular ferric blue or chromium oxide, or titanium oxide-coated mica with an organic pigment of the abovementioned type, and pearlescent pigments based on bismuth oxychloride.

The fat-soluble dyes are, for example, Sudan red, D&C Red 17, D&C Green 6, β-carotene, soybean oil, Sudan brown, D&C Yellow 11, D&C Violet 2, D&C Orange 5, quinoline yellow or annato. The water-soluble dyes are, for example, beetroot juice or methylene blue.

The composition of the invention can additionally comprise any additive conventionally used in cosmetics, such as antioxidants, fillers, preservatives, fragrances, neutralizing agents, preservatives, fragrances, neutralizing agents, thickeners, cosmetic or dermatological active principles, such as, for example, emollients, moisturizers, vitamins or sunscreens, and their mixtures. These additives can be present in the composition in a content ranging from 0 to 20% (in particular from 0.01 to 20%) of the total weight of the composition and better still from 0.01 to 10% (if present).

Of course, a person skilled in the art will take care to choose the optional additional additives and/or their amount so that the advantageous properties of the composition according to the invention are not, or not substantially, detrimentally affected by the envisaged addition.

The composition according to the invention can be manufactured by known processes used generally in the cosmetics or dermatological field.

The invention is illustrated in more detail in the following examples.

### Example 1

A mascara having the following composition was prepared:

| Carnauba wax | | 2.6 | g |
|---|---|---|---|
| Beeswax | | 3.3 | g |
| Paraffin wax | | 10.4 | g |
| Hydrogenated jojoba oil | | 0.2 | g |
| Hydrogenated palm oil | | 0.2 | g |
| Resin formed from polyamide with end | | | |
| ester groups, sold under the name | | | |
| “Uniclear ® 100” by Arizona Chemical | | 1 | g |
| 2-Amino-2-methyl-1,3-propanediol | | 0.8 | g |
| Triethanolamine | | 2.4 | g |
| Stearic acid | | 6.6 | g |
| Hydroxyethylcellulose | | 0.8 | g |
| Gum arabic | | 0.6 | g |
| Ethyl acrylate/methyl methacrylate (80/20) | | | |
| copolymer as a 50% AM aqueous dispersion | | | |
| (Daitosol 5 000 AD from Saito) | | 5 | g AM |
| Black iron oxide | | 7 | g |
| Preservatives | q.s. | | |
| Water | q.s. for | 100 | g |

This mascara is easily applied and adheres well to the eyelashes, during and after application; the latter are quickly made up. The mascara contributes instantaneous volumizing to the eyelashes.

### Example 2

A mascara having the following composition was prepared:

| Beeswax | | 7.1 | g |
|---|---|---|---|
| Hydrogenated jojoba oil | | 7.1 | g |
| Resin formed from polyamide with end | | | |
| ester groups, sold under the name | | | |
| “Uniclear ® 100” by Arizona Chemical | | 0.5 | g |
| Polybutylene | | 1 | g |
| Hydroxyethylcellulose/diallyldimethyl- | | | |
| ammonium chloride copolymer (Celquat LOR | | | |
| from National Starch) | | 3.8 | g |
| Glyceryl mono- and disterate (Tegin M | | | |
| from Goldschmidt) | | 2.1 | g |
| Glyceryl monostearate oxyethylenated | | | |
| with 30 mol of ethylene oxide (Tagat S | | | |
| from Goldschmidt) | | 5.5 | g |
| Black iron oxide | | 7 | g |
| Preservatives | q.s. | | |
| Water | q.s. for | 100 | g |

This mascara adheres well to the eyelashes, during and after application. It exhibits good instantaneous volumizing of the eyelashes.

### Example 3

a) A Microdispersion of Carnauba Wax having the Following Composition was Prepared:

| Carnauba wax | | 27 | g |
|---|---|---|---|
| Polyoxyethylenated (30 EO) glyceryl | | | |
| monostearate (Tagat S from Goldschmidt) | | 6.75 | g |
| Ethanol | | 10 | g |
| Water | q.s. for | 100 | g |

The wax and the surfactant were heated to 90° C. while homogenizing the mixture with moderate stirring. The water, heated to 90° C., was then incorporated while continuing to stir. The mixture was cooled to ambient temperature and the ethanol was added in order to obtain a wax microdispersion having a mean particle diameter of approximately 170 nm.

b) A Mascara having the Following Composition was Prepared:

| Wax microdispersion of example 1 | | 5 | g |
|---|---|---|---|
| Carnauba wax | | 1.5 | g |
| Beeswax | | 3.6 | g |
| Paraffin wax | | 11.5 | g |
| Hydrogenated jojoba oil | | 0.2 | g |
| Hydrogenated palm oil | | 0.2 | g |
| Resin formed from polyamide with end | | | |
| ester groups, sold under the name | | | |
| “Uniclear ® 100” by Arizona Chemical | | 0.5 | g |
| 2-Amino-2-methyl-1,3-propanediol | | 0.8 | g |
| Triethanolamine | | 2.4 | g |
| Stearic acid | | 6.6 | g |
| Hydroxyethylcellulose | | 0.8 | g |
| Gum arabic | | 0.6 | g |
| Ethyl acrylate/methyl methacrylate (80/20) | | | |
| copolymer as a 50% AM aqueous dispersion | | | |
| (Daitosol 5 000 AD from Saito) | | 5 | g AM |
| Black iron oxide | | 7 | g |
| Preservatives | q.s. | | |
| Water | q.s. for | 100 | g |

This mascara is easily applied and adheres well to the eyelashes, during and after application; the latter are quickly made up.

### Example 4

A mascara having the following composition was prepared:

| Carnauba wax | | 2.9 | g |
|---|---|---|---|
| Beeswax | | 3.6 | g |
| Paraffin wax | | 11.5 | g |
| Hydrogenated jojoba oil | | 0.2 | g |
| Hydrogenated palm oil | | 0.2 | g |
| Resin formed from polyamide with end | | | |
| ester groups, sold under the name | | | |
| “Uniclear ® 100” by Arizona Chemical | | 2 | g |
| 2-Amino-2-methyl-1,3-propanediol | | 0.8 | g |
| Triethanolamine | | 2.4 | g |
| Stearic acid | | 6.6 | g |
| Hydroxyethylcellulose | | 0.8 | g |
| Gum arabic | | 0.6 | g |
| Ethyl acrylate/methyl methacrylate (80/20) | | | |
| copolymer as a 50% AM aqueous dispersion | | | |
| (Daitosol 5 000 AD from Saito) | | 2.5 | g AM |
| Black iron oxide | | 7 | g |
| Preservatives | q.s. | | |
| Water | q.s. for | 100 | g |

This mascara is easily applied and adheres well to the eyelashes, during and after application; the latter are quickly made up. Instantaneous volumizing of the eyelashes is observed.

1. A composition comprising, in a physiologically acceptable aqueous medium, a wax-in-water emulsion and at least one first polyamide polymer with a weight-average molecular mass of less than 100 000 comprising a polymer backbone having hydrocarbonaceous repeat units which are provided with at least one nonpendent amide unit and at least one pendent fatty chain and/or at least one end fatty chain, which fatty chains are optionally functionalized, have from 8 to 120 carbon atoms and are bonded to these amide units.

2. The composition as claimed in claim 1, wherein the average molar mass of the first polymer is less than 50 000.

3. The composition as claimed in claims 1 or 2, wherein the fatty chains represent from 40 to 98% of the total number of amide units and of the fatty chains.

4. The composition as claimed in one of the preceding claims, wherein the fatty chains represent from 50 to 95% of the total number of the amide units and of the fatty chains.

5. The composition as claimed in one of the preceding claims, wherein the pendent fatty chains are bonded directly to at least one of the nitrogen atoms of the amide units.

6. The composition as claimed in one of claims 1 and 3 to 5, wherein the average molar mass of the first polymer ranges from 1 000 to 100 000, preferably from 1 000 to 50 000 and better still from 1 000 to 30 000.

7. The composition as claimed in one of the preceding claims, wherein the weight-average molar mass of the first film-forming polymer ranges from 2 000 to 20 000 and preferably from 2 000 to 10 000.

8. The composition as claimed in one of the preceding claims, wherein the end fatty chain or chains are bonded to the backbone via bonding groups.

9. The composition as claimed in claim 8, wherein the bonding groups are ester groups.

10. The composition as claimed in one of the preceding claims, wherein the fatty chain or chains have from 12 to 68 carbon atoms.

11. The composition as claimed in one of the preceding claims, wherein the first polymer is chosen from polymers of following formula (I) and their blends: 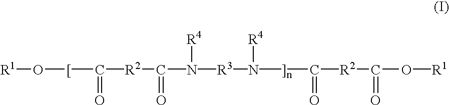
in which n denotes a number of amide units such that the number of ester groups represents from 10% to 50% of the total number of the ester and amide groups; R¹ is, in each case, independently an alkyl or alkenyl group having at least 4 carbon atoms; R² independently represents, in each case, a C₄ to C₄₂ hydrocarbonaceous group, provided that 50% of the R² groups represent a C₃₀ to C₄₂ hydrocarbonaceous group; R³ independently represents, in each case, an organic group provided with at least 2 carbon atoms, with hydrogen atoms and optionally with one or more oxygen or nitrogen atoms; and R⁴ independently represents, in each case, a hydrogen atom, a C₁ to C₁₀ alkyl group or a direct bond to R³ or another R⁴, so that the nitrogen atom to which both R³ and R⁴ are bonded forms part of a heterocyclic structure defined by R⁴—N—R³, with at least 50% of the R⁴ groups representing a hydrogen atom.

12. The composition as claimed in the preceding claim, wherein R¹ is a C₁₂ to C₂₂ alkyl group.

13. The composition as claimed in either of claims 11 and 12, wherein R² are groups having from 30 to 42 carbon atoms.

14. The composition as claimed in one of the preceding claims, wherein the first polymer is present in a content ranging from 0.01% to 10% by weight with respect to the total weight of the composition, preferably ranging from 0.05% to 5% by weight and better still ranging from 0.1% to 3% by weight.

15. The composition as claimed in any one of the preceding claims, which comprises at least one wax having a melting temperature of greater than 30° C. and ranging up to 120° C.

16. The composition as claimed in any one of the preceding claims, which comprises a wax chosen from the group formed by beeswax, lanolin wax, Chinese insect wax; rice wax, carnauba wax, candelilla wax, ouricury wax, cork fiber wax, sugar cane wax, Japan wax and sumac wax, montan wax, microcrystalline waxes, paraffin waxes, ozokerites, ceresin wax, lignite wax, polyethylene waxes, the waxes obtained by the Fischer-Tropsch synthesis, fatty acid esters and glycerides which are solid at 40° C., the waxes obtained by catalytic hydrogenation of animal or vegetable oils having linear or branched C₈-C₃₂ fatty chains, silicone waxes, fluorinated waxes, and their mixtures.

17. The composition as claimed in any one of the preceding claims, which comprises a wax having a hardness ranging from 0.05 MPa to 15 MPa.

18. The composition as claimed in any one of the preceding claims, wherein the wax is dispersed in the form of particles having a mean size ranging from 50 nm to 10 μm and preferably ranging from 50 nm to 3.5 μm.

19. The composition as claimed in any one of the preceding claims, wherein the wax is dispersed in the form of particles having a mean size ranging from 1 μm to 10 μm and preferably from 1 μm to 3.5 μm.

20. The composition as claimed in any one of claims 1 to 18, wherein the wax is dispersed in the form of particles having a mean size of less than 1 μm, preferably ranging from 50 nm to 500 nm.

21. The composition as claimed in any one of the preceding claims, wherein the wax is present in a content ranging from 0.1% to 50% by weight with respect to the total weight of the composition, preferably from 0.5% to 30% by weight and better still from 1% to 20% by weight.

22. The composition as claimed in any one of the preceding claims, which comprises a second film-forming polymer other than the first polymer.

23. The composition as claimed in claim 22, wherein the second film-forming polymer is chosen from the group formed by vinyl polymers, polyurethanes, polyesters, polyamides, polyureas and cellulose polymers.

24. The composition as claimed in claim 22 or 23, wherein the second film-forming polymer is dissolved in the aqueous phase.

25. The composition as claimed in claim 22 or 23, wherein the second film-forming polymer is in the form of particles in aqueous dispersion.

26. The composition as claimed in any one of claims 22 to 25, wherein [lacuna] second film-forming polymer is present in a content on a dry basis ranging from 0.1% to 60% by weight with respect to the total weight of the composition, preferably from 0.5% to 40% by weight and better still from 1% to 30% by weight.

27. The composition as claimed in any one of the preceding claims, which comprises an emulsifying surfactant.

28. The composition as claimed in claim 27, wherein the emulsifying surfactant is present in a content ranging from 2 to 30% by weight with respect to the total weight of the composition and better still from 5% to 15% by weight.

29. The composition as claimed in any one of the preceding claims, wherein the aqueous phase comprises an organic solvent which is miscible with water.

30. The composition as claimed in claim 29, wherein the water-miscible organic solvent is chosen from the group formed by lower monoalcohols having from 1 to 5 carbon atoms, glycols having from 2 to 8 carbon atoms, C₃-C₄ ketones and C₂-C₄ aldehydes.

31. The composition as claimed in claim 29 or 30, wherein the water-miscible organic solvent is chosen from the group formed by ethanol, isopropanol, propylene glycol, ethylene glycol, 1,3-butylene glycol and dipropylene glycol.

32. The composition as claimed in any one of claims 29 to 31, wherein the water-miscible organic solvent is present in a content ranging from 0.1% to 20% by weight with respect to the total weight of the composition, preferably ranging from 0.1% to 10%.

33. The composition as claimed in any one of the preceding claims, wherein the aqueous phase composed of water and optionally of water-miscible organic solvent is present in a content ranging from 1% to 95% by weight with respect to the total weight of the composition, preferably from 5% to 80% by weight and better still from 10% to 60% by weight.

34. The composition as claimed in one of the preceding claims, which additionally comprises at least one coloring material.

35. The composition as claimed in claim 34, wherein the coloring material is chosen from pigments, pearlescent agents, water-soluble dyes, and their mixtures.

36. The composition as claimed in claim 34 or 35, wherein the coloring material is present in a proportion of 0.01 to 50% of the total weight of the composition, preferably ranging from 0.01% to 30% by weight.

37. The composition as claimed in any one of the preceding claims, which comprises a thickening agent.

38. The composition as claimed in one of the preceding claims, which constitutes a composition for caring for or making up keratinous substances.

39. The composition as claimed in one of the preceding claims, which comprises at least one additive chosen from antioxidants, fillers, preservatives, fragrances, neutralizing agents, cosmetic or dermatological active principles, oils, and their mixtures.

40. The composition as claimed in one of the preceding claims, which is provided in the form of a mascara, an eyeliner, a product for the eyebrows, a product for the lips, a face powder or eyeshadow, a foundation, a product for making up the body, a concealer, a nail varnish, a product for caring for the skin or a product for the hair.

41. A mascara comprising the composition as claimed in any one of claims 1 to 40.

42. A cosmetic process for making up or caring for human keratinous substances, comprising the application, to the keratinous substances, of the cosmetic composition in accordance with one of claims 1 to 40.

43. Use of the composition as claimed in any one of claims 1 to 40 for producing an adherent layer on keratinous substances and/or for producing fast making up of keratinous substances.

44. Use of the mascara as claimed in claim 41 for rapidly thickening and/or for lengthening the eyelashes.

45. Use of the combination of at least one first polymer with a weight-average molecular mass of less than 100 000 and better still of less than 50 000, comprising a polymer backbone having hydrocarbonaceous repeat units which are provided with at least one nonpendent amide unit and at least one pendent fatty chain and/or at least one end fatty chain, which fatty chains are optionally functionalized, have from 6 to 120 carbon atoms and are bonded to these amide units, and of at least one wax, in a physiologically acceptable composition in the form of a wax-in-water emulsion, for producing an adherent layer on keratinous substances and/or fast making up of keratinous substances and/or for rapidly thickening and/or for lengthening the eyelashes.

46. Use as claimed in claim 45, wherein the first polymer is a polyamide comprising end groups comprising an ester group comprising a hydrocarbonaceous chain having from 10 to 42 carbon atoms.

47. Use as claimed in either of claims 45 and 46, wherein the first polymer exhibits a weight-average molecular mass ranging from 1 000 to 30 000.
